(19) United States
(12) Patent Application Publication
(10) Pub. No.: US 2010/0172953 A1
(43) Pub. Date: Jul. 8, 2010

(54) Biopolymer Based Implantable Degradable Devices

(75) Inventors: Christian Klein Larsen, Eiksmarka (NO); Therese Andersen, Sande i Vestfold (NO)

Correspondence Address: PATENT ADMINISTRATOR;FMC CORPORATION, 1735 MARKET STREET, PHILADELPHIA, PA, 19103, US

(73) Assignee: FMC CORPORATION, Philadelphia, PA (US)

(21) Appl. No.: 12/663,591

(22) PCT Filed: Jun. 13, 2008
(86) PCT No.: PCT/US08/66826

§ 371 Date: Mar. 10, 2010

Related U.S. Application Data
(60) Provisional application No. 60/943,800, filed on Jun. 13, 2007.
(60) Provisional application No. 60/943,787, filed on Jun. 13, 2007.
(60) Provisional application No. 61/013,216, filed on Dec. 12, 2007.
(60) Provisional application No. 61/013,223, filed on Dec. 12, 2007.

(51) Int. Cl. A61K 38/18; A61F 2/00; A61P 19/04

(52) U.S. Cl. 424/423; 514/12

(57) ABSTRACT

Implantable degradable devices for tissue repair or reconstruction comprising biopolymers, as well as to methods of manufacture and use thereof. The implantable device is formed by the application of pressure and the device may include up to about 65% by weight of water, based on the total weight of the implantable degradable fastening device. Methods for making implantable, degradable devices from biopolymers by application of pressure are also disclosed. The invention provides the ability to customize the device in various ways to influence properties such as mechanical strength, degradation rate and swellability.

### FIELD OF THE INVENTION

The present invention is directed to implantable degradable devices for tissue repair or reconstruction comprising biopolymers, as well as to methods of manufacture and use thereof.

### BACKGROUND OF THE INVENTION

Use of implantable degradable devices, such as devices made of erodible/enzymatically degradable biopolymers, e.g., alginate, chitosan, hyaluronate or their derivatives will minimize or eliminate the need for a second surgery to remove the implanted device. It may also eliminate or reduce the occurrence of complications during a potential second surgery and it should reduce the likelihood of secondary fractures resulting from the stress-shielding effect or the presence of screw holes that serve as stress concentrators. Use of degradable devices will also eliminate the cost related to secondary surgeries since such devices need not be removed once implanted.

Some bioabsorbable products on the market consist of polymers that release degradation products not favorable for the healing area. Examples of bioabsorbable materials used in existing degradable fixation products are polyhydroxyacids, e.g. polylactides, polyglycolides and their copolymers, and polycarbonates. The degradation products from polyhydroxyacids induce an unfavorable lowered pH value around the healing area. An effect of this is prolonged inflammatory response and reversal of an initial healthy tissue response.

Alginate is a widely used material for tissue regeneration and cell immobilization, for example, in the form of hydrogels or porous scaffolds. Chitosan is also a common biopolymer in implantable biomaterials, and it is known from the literature to enhance osteogenesis and is of special interest for scientists working in the orthopedic area. Hyaluronate is a biopolymer naturally occurring in the human body as the second most abundant after collagen in the extracellular matrix (ECM). Hyaluronate is also an important component of articular cartilage and it contributes to tissue hydrodynamics, movement and proliferation of cells, and participates in a number of cell surface receptor interactions.

Zhong et al., U.S. Pat. No. 6,368,356, discloses medical devices comprising hydrogel polymers with ionic crosslinks having improved mechanical strength with at least two segments that degrade in vivo at different rates. The different segments differ in their type of crosslinking, ionic versus covalent, or, alternatively the segments are not biodegradable.

Luzio et al., U.S. Pat. No. 5,531,716, discloses medical devices subjected to triggered disintegration. The medical devices comprise ionically crosslinked polymers that have sufficient mechanical strength to serve as a stent, catheter, cannula, plug or constrictor. The methods presented to create the materials involve forcing the crosslinkable polymer through a shaping die into a crosslinking bath, use of molding compositions with the crosslinkable polymer in solution, or use of materials wherein the crosslinking ion is in an insoluble or slowly soluble form, and additives are included to cause dissolution of the crosslinking ion. The created gel can be further developed, crosslinked and/or shaped by soaking in a solution of a crosslinking ion. Also required is a triggered disintegration of the device induced by administering or triggering release of an agent which displaces the crosslinking ion through the diet, parenteral feeding or an enema, administering the agent directly onto the device in an aqueous solution or encapsulating the agent in the device.

Teoh et al., U.S. patent application publication no. US 2007/0083268 A1, discloses bioabsorbable plug implants and methods for bone tissue regeneration. The bioabsorbable plug implants comprise a first portion and a second portion extending outwardly from the first portion, the first and second portions being formed from expandable material. It is mentioned that any bioabsorbable material known in the art suitable for the construction of the plug implant can be used. In the method for bone tissue regeneration of the device may be inserted into a defect or gap of a bone.

Ashammakhi and Törmälä in International patent application publication no. WO 2005/009496, present an implant device for bone fixation or augmentation in a mammalian body to enhance the mechanical strength of a fracture.

### SUMMARY OF THE INVENTION

In a first embodiment, the present invention relates to degradable devices made from biopolymers and derivatives thereof and to implantable devices including at least one degradable biopolymer or a derivative thereof, e.g., alginate, chitosan, hyaluronans or their derivatives. The devices provide a combination of degradability and biocompatibility with physical properties suitable for use of the devices as implants. Exemplary devices are devices including one or more biopolymers. The use of such degradable biopolymers minimizes or eliminates the need for a second surgery to remove the implant, thereby eliminating the additional cost and potential complications of such a second surgery and should reduce the likelihood of secondary fractures resulting from the stress-shielding effect or the presence of screws holes that serve as stress concentrators.

In other embodiments, the present invention relates to methods for the fabrication of the devices of the present invention. Such methods involve the exertion of pressure on a partially or fully hydrated biopolymer and, optionally, at least partially drying the biopolymer. Such methods include, for example, extrusion, milling and molding.

### DESCRIPTION OF FIGURES

FIG. 1 shows the test probe and a specially designed jig to allow for injection of water for measurement of break force and breaking time of hydrated samples using the Texture Analyzer from Stable Micro Systems (TA-XT2i).

FIG. 2 shows the breaking time under load after addition of water to dry alginate bolts prepared by air drying and freeze drying, respectively, as a function of the dry break strength.

FIG. 3 shows a test jig used to measure breakage strength of the bolts

### DETAILED DESCRIPTION OF THE INVENTION

The present invention is directed to an implantable degradable device comprising biopolymers, as well as to methods of manufacture and use thereof. Biopolymers include polymers that are produced by living organisms, as well as materials derived from biopolymers by some type of synthetic modification of the material that was produced by a living organism. Some examples of such synthetic modification processes are described below. Classes of suitable biopolymers include polysaccharides, polypeptides and polypeptides covalently bonded to polysaccharides in any desired ratio.

As used herein, “degradable” refers to the device of the present invention wherein the device naturally disappears over time in vivo from or in accordance with any biological or physiological mechanism, such as, for example, erosion including bioerosion, degradation, dissolution, chemical depolymerization including at least acid- and base-catalyzed hydrolysis and free radical induced depolymerization, enzymatic depolymerization, absorption and/or resorption within the body. As a result, the degradable devices of the present invention do not require surgical removal.

The use of biopolymers as the degradable material for fixatives will be beneficial compared to the commonly used synthetic polymers due to surface properties. As the surfaces of many synthetic polymers are hydrophobic this will hinder cell growth, whereas hydrophilic biopolymers may promote cell proliferation and cell differentiation. Additionally, further modification of synthetic polymers may be necessary to provide the required functional groups.

Examples of the biopolymers that may be used in the present invention include alginates, chitosans, hyaluronates their derivatives and mixtures thereof. None of these biopolymers are known to cause unfavorable conditions for formation of new tissue upon degradation. Degradable medical attachment devices of the invention comprising biopolymers from any of the above listed biopolymers are suitable for tissue repair or reconstruction by, for example, attachment of damaged tissue for regrowth of the tissue.

Ultrapure biopolymers having sufficient purity to render such biopolymers suitable for implantation without causing inflammatory responses should be used. Ultrapure biopolymers have a reduced content of endotoxins. By reduced endotoxin content, it is meant that the endotoxin, protein and heavy metal content of the biopolymers used to prepare the device and the endotoxin content of the medical device together must not exceed, for example, the U.S. Food and Drug Administration recommended endotoxin contents for implantable medical devices. The current regulatory guidelines establish that a device may not release to the patient more than 350 EU (5 EU/kg).

When alginate is used as the biopolymer, the gelling cations that may be present will be exchanged with non-gelling ions over time, which makes the polymer soluble. Soluble alginate will be depolymerized by acid- or base-catalyzed hydrolysis, or free radicals. When the alginate has been depolymerized to a lower molecular weight, it is naturally excreted from the body through the kidneys. When chitosan is used as the biopolymer, it will undergo enzymatic hydrolysis mediated by lysozymes present in mammalians in saliva, tears, blood serum and in interstitial fluid. Additionally anions will be exchanged over time if the chitosan is ionically crosslinked. When hyaluronate is used as the biopolymer it will be enzymatically degraded by hyaluronidases present in mammalians in tissues and cells, blood plasma, synovial fluid and urine. The device of the invention can be designed to retain the needed strength for a sufficient time period after insertion and then gradually disappear, e.g., degrade/bioabsorb, as the healing process progresses. None of the degradation products are known to induce any undesired effects for the newly formed tissue or within the human or mammalian body.

Alginates are salts of alginic acid. Alginates are a family of non-branched binary copolymers of 1→4 glycosidically linked β-D-mannuronate (M) and α-L-guluronate (G) monomers. The relative amount of the two uronate monomers and their sequential arrangement along the polymer chain vary widely, depending on the origin of the alginate. Alginate is the structural polymer in marine brown algae such as Laminaria hyperborea, Macrocystis pyrifera, Lessonia nigrescens and Ascophyllum nodosum. Alginate is also produced by certain bacteria such as Pseudomonas aeruginos and Azotobacter vinelandii. The ratio of mannuronate and guluronate varies with factors such as seaweed species, plant age, and part of the seaweed (e.g., stem, leaf). The uronic acid residues are distributed along the polymer chain in a pattern of blocks, where homopolymeric blocks of G residues (G-blocks), homopolymeric blocks of M residues (M-blocks) and blocks with alternating sequence of M and G units (MG-blocks) co-exist. The alginate molecule cannot be described by the monomer composition alone. Composition and sequential structure together with molecular weight and molecular conformation are the key characteristics of alginate in determining its properties and functionality.

Examples of the alginate include alginate having a G content greater than 50%, a G content greater than 60%, a G content greater than 70%, a G content greater than 80%, and a G content greater than 90% and mixtures thereof. Additional examples include an alginate having an M content of greater than 50%, an M content greater than 60%, an M content greater than 70%, and an M content greater than 80% and mixtures thereof. Mixtures of alginates having such G content and M content may also be used. Examples of the alginate include alginate having a molecular weight less than 500 kDa. Suitable alginates have a molecular weight greater than 4,000 Daltons. Products may contain any suitable amount of alginate, for example, at least 85% by weight of alginate, at least 90% by weight of alginate, at least 95% by weight of alginate, or 100% by weight of alginate. It has been found that decreasing the G content of the alginate relative to the M content produces stronger dried devices.

Chitin is a linear polysaccharide comprising β-(1→4)-linked 2-acetamido-2-dexoy-D-glucopyranose (GlcNAc) and 2-amino-2-deoxy-D-glucopyranose (GlcN). Chitin is present in nature as the structural element in the exoskeleton of crustaceans (crabs, shrimps, etc.). Chitosan is a fully or partially N-deacetylated derivative of chitin. Chitin consists nearly entirely of β-(1→4)-linked 2-acetamido-2-dexoy-D-glucopyranose (GlcNAc). Commercially chitosan is made by alkaline N-deacetylation of chitin. The heterogeneous deacetylation process combined with removal of insoluble compound results in a chitosan product which possesses a random distribution of GlcNAc and GlcN units along the polymer chain. The amino group in chitosan has an apparent pKₐ-value of about 6.5 and at a pH below this value, the free amino group will be protonized so the chitosan salt dissolved in solution will carry a positive charge. Accordingly, chitosan is able to react with negatively charged components, it being a direct function of the positive charge density of chitosan. The positive charge gives the chitosan bioadhesive properties.

Hyaluronate is a linear polymer that is composed of glucuronate and N-acetylglucosamine monomers linked alternately by β(1→3) and β(1→4) glycosidic bonds. The polymer is an important part of the extracellular matrix, for example is it a major component of the synovial fluid. It was found to increase the viscosity of fluids and along with lubricin, it is one of the fluid's main lubricating components as the coiled structure can trap approximately 1000 times its weight in water. Hyaluronate is also an important component of articular cartilage and a major component of skin, where it is involved in tissue repair.

Commercially available hyaluronate is usually made by fermentation from e.g. Streptococcus zooepidemicus or derived from avian (chicken or rooster) combs. The available molecular weights of commercially available hyaluronates are less than 5000 kDa and will be suitable for this invention.

The biopolymers can be tailored to the specific application by choosing the appropriate chemical composition of the biopolymers used and also by modification of the biopolymers if desired. Biopolymer derivatives or modified biopolymers with altered properties or functionalities such as crosslinking capability, solubility, rate of biodegradability, the ability to bind, for example, specific cells, pharmaceuticals or peptides, are included within the scope of the invention. Modified polysaccharides, for example, peptide-coupled polysaccharides are prepared by means known in the art. For example, modified alginates are disclosed in U.S. Pat. No. 6,642,363 (Mooney). Peptide-coupled polysaccharides are preferred for use for example in immobilizing cells to promote cell proliferation, viability and cell differentiation. Peptide-coupled polysaccharides are preferably employed in combination with non-modified polysaccharides.

Modified polysaccharides may include synthetic analogues of polysaccharides formed by covalent bonding onto the polysaccharide, polysaccharides modified by enzymatic modification, e.g. epimerization of alginates, as well as oxidation of polysaccharides. Covalent bonding may be used to attach a variety of materials including peptide sequences, sugar units, and hydrophobic groups such as thiol groups and alkyl chains (WO/2003/080135 or Kang et al., Polymer Bulletin, 47 (5), 429-435, 2002 respectively).

Modified polysaccharides formed by covalent bonding may be formed by covalently linking the polysaccharide to a polymer backbone. Preferred linked polysaccharide groups are alginates or modified alginates containing functional sites. The polysaccharide, particularly alginate, when present as side chains on the polymer backbone, may include side chains at the terminal end of the backbone, thus being a continuation of the main chain. The modified polysaccharides and modified alginates exhibit controllable properties depending upon the ultimate use thereof. One example of modified alginates can be found in U.S. Pat. No. 6,642,363 (Mooney et al.), the disclosure of which is hereby incorporated by reference for a description of such materials and methods for making them. Mooney et al. discloses modified alginates, methods of preparation and uses thereof such as cell transplantation matrices, preformed hydrogels for cell transplantation, non-degradable matrices for immunoisolated cell transplantation, vehicles for drug delivery, wound dressings and replacements for industrially applied alginates.

Modified polysaccharides such as modified alginates may also be prepared by covalently bonding to add a biologically active molecule for cell adhesion or other cellular interaction. Crosslinked modified alginates with the biologically active molecules in a three-dimensional environment are particularly advantageous for cell adhesion, thus making such alginates useful as cell transplantation matrices. In some embodiments, the modified alginate is a biologically active molecule for cell adhesion or other cellular interaction, which is particularly advantageous for maintenance, viability, proliferation, mobility and differentiation.

Modified alginates can also be prepared using an approach combining chemical and enzymatic techniques. One example of this approach can be found in International patent application publication no. WO 06/051421 A1. The starting alginate can have varying amounts of M and G which may be grouped in varying structural arrangements of MM, GG, and/or MG blocks. A chemical reaction step will lead to substituents reacted on the M and G residues of the alginate as applicable. The enzymatic step will change the amount of M and G in the alginate by converting a desired number of M residues to G residues. For example, the amount of G is increased by converting MM blocks to MG or GG or converting MG blocks to GG.

In some embodiments, the biopolymer, e.g. alginate comprises one or more cell adhesion peptides covalently linked thereto. In some embodiments, the alginate comprises one or more cell adhesion peptides covalently linked thereto. Suitable modified alginates containing cell adhesion peptides comprising RGD include, but are not limited, to Novatach RGD (NovaMatrix, FMC BioPolymer, Oslo, Norway) and those disclosed in U.S. Pat. No. 6,642,363, which is hereby incorporated by reference for the description of these materials. Peptide synthesis services are available from numerous companies, including Commonwealth Biotechnologies, Inc. of Richmond, Va., USA. Chemical techniques for coupling peptides to the alginate backbones may be found in U.S. Pat. No. 6,642,363.

Coupling of the cell adhesion molecules to the alginate can be conducted utilizing synthetic methods which are in general known to one of ordinary skill in the art. A particularly useful method is by formation of an amide bond between the carboxylic acid groups on the alginate chain and amine groups on the cell adhesion molecule. Other useful bonding chemistries include those discussed in Hermanson, Bioconjugate Techniques, p. 152-185 (1996), particularly by use of carbodiimide couplers, DCC and DIC (Woodward's Reagent K). Since many of the cell adhesion molecules are peptides, they contain a terminal amine group for such bonding. The amide bond formation is preferably catalyzed by 1-ethyl-3-(3-dimethylaminopropyl)carbodiimide (EDC), which is a water soluble enzyme commonly used in peptide synthesis.

Examples of modified alginates may be found in, “Dual Growth Factor Delivery and Controlled Scaffold Degradation Enhancement in vivo Bone Formation by Transplanted Bone Marrow Stromal Cells,” Simmons, C. A. et al., Bone 35 (2004), pp. 562-569, and “Regulating Bone Formation via Controlled Scaffold Degradation,” E. Alsberg, et al., J. Dent. Res. 82(11), pp. 903-908 (2003). Examples of oxidized alginates can be found, for example, in European patent application publication no. EP 0 849 281.

Modified biopolymers may also be made by partially or fully crosslinking the biopolymers. A variety of different types of biopolymers may be prepared including, for example, non-crosslinked biopolymers, ionically crosslinked biopolymers or covalently crosslinked biopolymers. The degree of crosslinking can be stoichiometric or sub-stoichiometric, as desired to obtain the particular properties sought for a particular device or part of a device. In this manner, partial crosslinking can be employed as one method for providing a controlled rate of degradation of the device or a portion thereof. The rate of degradation or resorption of the biopolymer system may be controlled by varying the degree of cross-linking and the molecular weight of the components of the device using any suitable technique, one illustrative technique being described in, for example, Kong, et al “Controlling rigidity and degradation of alginate hydrogels via molecular weight distribution.” Crosslinking agents may optionally be present in an amount sufficient to saturate the biopolymer to 0.001% to 200%.

One method of crosslinking is ionic crosslinking. The crosslinking ions are generally classified as anions or cations. Appropriate crosslinking ions include but are not limited to cations comprising an ion selected from the group consisting of calcium, magnesium, barium, strontium, boron, beryllium, aluminum, iron, copper, cobalt, lead, and silver ions, and anions selected from the group consisting of phosphate, citrate, borate, succinate, maleate, adipate and oxalate ions. More broadly the anions are derived from polybasic organic or inorganic acids. Preferred crosslinking cations are calcium, iron, and aluminum ions. The most preferred crosslinking cations are calcium and iron ions. The most preferred crosslinking anion is phosphate.

Appropriate agents that displace a crosslinking ion include, but are not limited to ethylene diamine tetraacetic acid, ethylene diamine tetraacetate, citrate, organic phosphates, such as cellulose phosphate, inorganic phosphates, as for example, pentasodium tripolyphosphate, mono and dibasic potassium phosphate, sodium pyrophosphate, and phosphoric acid, trisodium carboxymethyloxysuccinate, nitrilotriacetic acid, maleic acid, oxalate, polyacrylic acid, sodium, potassium, calcium and magnesium ions. Preferred agents are citrate, inorganic phosphates, sodium, potassium and magnesium ions. The most preferred agents are inorganic phosphates and magnesium ions.

In one embodiment, preferred products are uncrosslinked or substantially uncrosslinked. In other embodiments, products are not ionically crosslinked or not substantially ionically crosslinked. For example, when referring to materials that are not substantially crosslinked, the degree of crosslinking may be selected for the purpose of stabilizing the material rather than a substantially greater amount which would cause gelation of the material. In alginates, it is believed that the presence of small amounts calcium ions form more stable aggregates of the alginates without substantial gelation of the alginate. In this manner, the implanted device may be stabilized against the influence of materials that it may contact in the body to minimize alteration of the device in use by such materials. Examples of the invention below indicate behavior of the devices under simulated implantation conditions using Ringers and Hank's balanced solutions. Some crosslinking may occur in the implant, once implanted, due to the presence of crosslinking cations in body fluids.

For example, alginates may be crosslinked using divalent cations. As used herein, “100% saturation” of the alginate molecule is considered to be 1 mole divalent cation per 2 moles uronate (D-mannuronate and L-guluronate). Alginates create heat stable gels at physiologic conditions when divalent cations as e.g. calcium, strontium or barium are present. Suitable crosslinking agents for the biopolymers of the invention may contain divalent or trivalent cations or water soluble salts containing phosphate or citrate. Suitable cations may include, but are not limited to, calcium, barium, lead, manganese, cobalt, nickel, iron, zinc, copper, aluminum, citrate, holmium and phosphate.

Chitosan deacetylation protects the polymer from enzymatic degradation. Thus, varying the degree of chitosan deacetylation can modify the rate of biodegradation of implanted chitosan-containing devices by lysozymes. Chitosans with higher degrees of deacetylation are also more resistant to random depolymerization by acid hydrolysis due to a protective effect of the positive charge. Examples of the chitosan include chitosan with a degree of deacetylation in the range of 40% to 100%. Suitable molecular weights are in the range 10 kDa to 1000 kDa. Blends of alginates and chitosans may be particularly advantageous since the anionic alginates may interact with the cationic chitosans to form a more stable matrix of material.

In one aspect of the present invention, anionic and cationic biopolymers are mixed or blended to form the biopolymer used in the devices of the present invention. It has been found, for example, that blending of anionic and cationic biopolymers at varying ratios can be employed to customize at least the strength, degradation and swelling properties of the resultant device. Depending on the particular use desired for a particular device, it may be beneficial to customize these properties for that use. Blends of hyaluronate and chitosan may be particularly advantageous since the anionic hyaluronate may interact with the cationic chitosans to form a more stable matrix of material. Blends typically contain from about 25 to about 75% by weight of the cationic polymer, based on the total weight of the cationic and anionic polymers, and, more preferably, contain from about 35 to about 65% by weight of the cationic polymer, most preferably, from about 45 to about 55% by weight of the cationic polymer, based on the total weight of the cationic and anionic polymers.

The implantable devices of the present invention are characterized by use of a step of applying pressure to the device during the fabrication process. The application of pressure during fabrication provides certain advantages to the device as discussed in detail below and in the examples appended hereto. In certain embodiments, the implantable device of the present invention may have an elongated body. In certain embodiments of the present invention, the implantable device of the present invention can be a screw, plug, bolt, anchor or pin that can be used for fixation of any portion of body tissue (e.g., muscle, bone, cartilage, tendon, etc.). The device of the invention may be designed to withstand one or more of torque, compressive, tensile and bending forces. A thread design may easily be made on the device as well. When the device of the invention is a screw, it may be a fully-threaded screw, i.e. a screw with threads along the entire length of the device, or it may be a partially threaded screw with threads located only on a proximal or distal part of the screw. These devices do not require surgical removal. Eliminating the insertion of a non-biologic implant will have several advantages. Removal of the implant and a second surgery will not be necessary, and the establishment of a new growing tissue will not be inhibited. Additionally a degradable implant will save both time and costs.

The terms, “fixation” and “fixative” refer to devices that are used to position or fix tissue in a desired position, location, orientation or attach or position tissue relative to other tissue, e.g. by attaching two tissues together or supporting two tissues in relationship to one another, including, but not limited to by attachment to the tissue, support of the tissue, or a combination thereof. Fixation of tissue does not necessarily require a load-bearing device and thus in some case, fixatives will not be load-bearing when implanted. For example, in the case of a plug, the plug may be implanted to ensure that materials are maintained in place during a healing period, in which case the plug may not have to bear a load. In another example, the plug may be used to provide a substrate into which a load bearing device may be incorporated, e.g. a plug with a load-bearing screw threaded into it.

The devices of the present invention may be load-bearing. Thus, some devices of the present invention will have sufficient strength and structural integrity to bear a load in use. By “load-bearing” is meant that the device is fabricated to have sufficient strength and/or structural integrity to bear a load that will be exerted on the device once it is implanted. Load-bearing may refer to a variety of different properties of the device such as its ability to withstand compressive, tensile, torsional and bending forces. A particular device may be able to withstand different levels of these various forces, depending on what is required for the particular use for which that device is destined.

The fixative may also be load-bearing and could be a screw which threadably engages tissue such as bone. In another example, the fixative can be a plug which fills a gap or hole in a tissue or fills corresponding gaps or holes in two or more tissues to position the tissues relative to one another. Fixation devices or fixatives include, but are not limited to fastening devices.

The device of the invention can be solid through or hollow through parts of the material or through the whole material. Alternatively, the device may include a partially hollow degradable biopolymer portion. The devices of the present invention may, in some embodiments have a rotationally symmetric shape. The degradation properties of the device may be customized by one or more of the additives, treatments and/or structures described above such that the device may immediately begin to degrade, may exhibit sustained degradation or may have delayed degradation. Also, various parts of the device may be tailored to have different degradation rates and/or immediate, sustained or delayed degradation.

Another aspect of the device of the present invention is that it is degradable. Thus, over a period of time, the device should degrade by one or more of the various mechanisms described above. Preferably, the device degrades over a period of 1-6 months, and more preferably, over a period of 2-4 months, or longer. In such case, the device should maintain its important characteristics (e.g. ability to bear a load) during the time period specified. The degradation rate of the device can be tailored using many of the fabrication methods, treatment processes, materials, structures and combinations thereof, which have been described herein.

Suitably, the device of the invention is sterilized preferably by γ-irradiation, E-beam, ethylene oxide, autoclaving, alcohol treatment, supercritical CO₂, or contacting with NOx gases or hydrogen gas plasma sterilization. A suitable sterilization should be employed which does not adversely affect the properties of the device in a significant, detrimental manner.

The swelling properties of the devices of the present invention may be customized for a particular use. In some embodiments, a relatively high swellability may be desired, for example, to provide a friction fit or force fit between the implant and the tissue. A plug implanted in a hole or gap in a bone may be retained in position by swelling of the plug to provide a tight fit with the bone. In some embodiments, swelling may be beneficial for triggering tissue regeneration by exertion of pressure on the area where tissue regeneration is desired. In other embodiments, a relative low swellability may be desired such that the device substantially retains its original size when implanted. In most embodiments a swellability of not more than 25% of the original size of the device, is desired. More preferably, for devices requiring lower swellability, swellability may be from 0% to 15%, and most preferably from 0% to 10%.

The swellability of the device can be influenced, for example, by coating a core of the device with fibers in order to retard swelling. Swelling can also be influenced by the method of making the device, the biopolymers used to make the device, post treatment processes and drying methods. In this manner, the swelling properties can be customized for a particular device or application, as desired.

Salts can be added to pastes comprising charged biopolymers such as hyaluronate and chitosan to control the hydration and/or degradation rates of the dried implanted materials. Adding salts as e.g. sodium chloride or calcium chloride will shield charges on both polymers preventing them from interacting with each other and thereby produce a less stable material which can degrade faster.

In some embodiments, the devices of the present invention may contain degradable biopolymer, as well as one or more of an uncrosslinked degradation controlling agent, an imaging agent, a gelling ion, an alcohol a tissue regenerative additive, a cell adhesion peptide sequence, or a pharmaceutically active agent selected from, but not limited to, a growth factor agent, an antiseptic, an anticoagulant, an antibiotic, an anti-inflammatory, a pain-killer, a chemotherapeutic agent, cells and an anti-infective agent, a protein or a drug to modify the properties of the device. The device may also contain one or more other therapeutic agents selected from enzymes, transcription factors, signaling molecules, internal messengers, second messengers, kinases, proteases, cytokines, chemokines, structural proteins, interleukins, hormones, pro-coagulants, agents that promote angiogenesis, agents that inhibit angiogenesis, immunomodulators, chemotactic agents, agents that promote apoptosis, agents that inhibit apoptosis, and mitogenic agents. The cell adhesion peptide sequence may be a biologically active molecule for promoting or causing cell adhesion or other cellular interaction. Combinations of two or more different cell adhesion peptide-linked biopolymers for example in biostructures, beads or hydrogels may provide particularly useful advantages for repairing, reconstructing and treating conditions of tissue. These additional materials may be provided to the device of the present invention in any suitable manner, for example, by being directly mixed into the biopolymer, as part of or as a coating on the device, as a filler in hollow portions of the device as described herein or as a filler contained in a suitable vehicle, e.g. a biopolymer hydrogel, located in hollow portions of the device.

Biologically active molecules for cell adhesion or other cellular interaction are well known and widely recognized and available. U.S. Pat. Nos. 4,988,621, 4,792,525, 5,965,997, 4,879,237, 4,789,734 and 6,642,363, which are incorporated herein by reference, disclose numerous examples. Suitable peptides include, but are not limited to, peptides having about 10 amino acids or less. In some embodiments, cell adhesion peptides comprise RGD, YIGSR (SEQ ID NO:1), IKVAV (SEQ ID NO:2), REDV (SEQ ID NO:3), DGEA (SEQ ID NO:4), VGVAPG (SEQ ID NO:5), GRGDS (SEQ ID NO:6), LDV, RGDV (SEQ ID NO:7), PDSGR (SEQ ID NO:8), RYVVLPR (SEQ ID NO:9), LGTIPG (SEQ ID NO:10), LAG, RGDS (SEQ ID NO:11), RGDF (SEQ ID NO:12), HHLGGALQAGDV (SEQ ID NO:13), VTCG (SEQ ID NO:14), SDGD (SEQ ID NO:15), GREDVY (SEQ ID NO:16), GRGDY (SEQ ID NO:17), GRGDSP (SEQ ID NO:18), VAPG (SEQ ID NO:19), GGGGRGDSP (SEQ ID NO:20) and GGGGRGDY (SEQ ID NO:21) and FTLCFD (SEQ ID NO:22). Cell adhesion peptides comprising RGD may be in some embodiments, 3, 4, 5, 6, 7, 8, 9 or 10 amino acids in length. Biologically active molecules for cell adhesion or other cellular interaction may include EGF, VEGF, b-FGF, FGF, TGF, TGF-β or proteoglycans.

When using “RGD peptides”, those peptides containing the RGD motif such as GGGGRGDY, GGGGRGDSP, GRGDSP, the interaction is dependent upon the way the RGD sequence is presented to the cells, for example, the concentration and/or the orientation.

A plasticizer may also be employed in the device of the present invention. When a plasticizer is employed in the device of the present invention, an amount of 0.01% to 70% by weight of the biopolymer may be employed. More preferably, 0.01% by weight to 50% by weight of the plasticizer, based on the weight of the biopolymer may be employed. Alternatively, an amount of 0.01% by weight to 25% by weight of plasticizer, based on the weight of the biopolymer, may be employed. Suitable plasticizers include, for example, at least one of glycerin, sorbitol, ethylene glycol, propylene glycol, and polyethylene glycol.

The present invention also relates to a method for making a degradable fastening device by forming the device from at least one biopolymer. The device may include any one or more of the additives or modifications discussed herein. Such devices may include screws, bolts, anchors, plugs, pins, or rods.

In general terms, the method of the present invention involves to the application of pressure to a partially hydrated biopolymer or biopolymer derivative-containing material to form a degradable pre-shaped device, such as a fixative device having the desired shape prior to implantation. By “pre-shaped” is meant that the device is shaped to substantially its final shape prior to implantation into the body. Some swelling or shrinkage of a pre-shaped device may occur upon implantation and thus devices that may undergo some shrinkage and/or swelling, particularly when exposed to body fluids, are still considered to be pre-shaped so long as they retain substantially their original shape after swelling or shrinkage. Pressure may be applied, for example, by molding, extrusion or other suitable processes. The application of pressure may compress, compact or densify the material. Also, some de-aeration of the material may occur as a result of the application of pressure due to compression of the material. It has been observed that in some embodiments using biopolymers, the application of pressure may cause a transition to a more transparent material, perhaps due to more uniform hydration of the material as a result of compression. Thus, when applying pressure to biopolymers, in some embodiments, sufficient pressure should be applied to provide a substantially homogeneous material which is transparent. By substantially homogeneous is meant that the hydration of the material is nearly uniform throughout the material once sufficient pressure has been applied.

The material may be partially or fully hydrated prior to application of pressure with higher degrees of hydration being preferred since a higher degree of hydration appears to provide a material of greater strength in the formed device.

The device may optionally be dried. Any conventional drying process may be used although, in some instances, controlled drying may be desirable for a variety of reasons such as controlling the shape and/or size of the final device. Preferred drying methods include air drying and freeze drying. It has been found that use of a particular drying process may influence the final properties of the device and thus selection of a drying process may be employed for device customization. For example, the breaking strength of the device can be altered by selection of a particular drying process, as shown in the examples below. Also, freeze drying can be used to increase the porosity of the device or enhance the degradation rate of the device. The devices of the present invention may typically have densities of from about 0.6 to about 1.5 mg/cm³, and, more preferably, have densities of about 0.8 to about 1.3 mg/cm³.

The water content of the material prior to application of pressure may vary over a wide range. In practice, the water content may depend on such factors as the degree of hydration that is desired for a particular material, as well as the flowability of the material that may be required for processing. Thus, water contents of 40-65% by weight are preferred for the materials of the present invention that are fed to the step of applying pressure since at these water contents, the material is best-suited for processing and can be handled in an efficient manner. In addition, it has been found that use of water contents of about 65% or higher may increase porosity of a freeze dried device. Thus, high water contents may be used to fabricate devices for which high porosity is desired. However, higher porosity was associated with lower breaking strength indicating that for load bearing applications, steps may be need to be taken to increase strength, e.g. fabrication of devices having larger dimensions and thus higher breaking strengths. Also, use of lower water contents may be a way to reduce shrinking of the product, upon drying.

Preferred products may thus have a water content of up to about 65% by weight, based on the total weight of the device, if no drying step is employed. Thus, devices of the present invention may comprise from 35%-100% solids, by weight, based on the total weight of the device, more preferably, from 40-100% solids. The solids content of the device will generally comprise, in large part, the biopolymer, but may also comprise, for example, plasticizers and other additives as discussed herein.

Dried devices will typically have solids contents of 80-100% by weight, more preferably, at least 88-95% by weight, based on the total weight of the device. Dried devices are preferred for load-bearing applications since dried devices appear to exhibit greater strength than materials which are not dried and thus are particularly suitable as fixatives where load-bearing is required.

The devices of the invention may have a water:biopolymer ratio of 2:10 to 0.01:10, more preferably, a water:biopolymer ratio of 1.5:10 to 0.5:10.

Devices which have not been subjected to a drying step and thus have higher water contents on the order of 15-65% by weight, more preferably, 40-60% by weight, are particularly useful for non-load-bearing applications of the present invention such as non-load bearing fixatives, promotion of tissue regrowth and for delivery of therapeutic agents or other materials which may be incorporated into the devices of the present invention as disclosed herein. These so-called wet devices still exhibit a relatively high content of solids, which are primarily or completely biopolymers, on the order of 35-85% by weight, more preferably 40-60% by weight. These materials are preferably not substantially gelled or crosslinked though some minor amounts of crosslinking agents or ions may be employed to stabilize the wet devices as discussed herein, if desired.

For the wet devices, the step of applying pressure to the paste may be employed, for example, to modify the hydration and/or degradation rate of the resultant wet device and/or to modify the release properties of the device by altering the release rate of incorporated materials such as therapeutic agents.

One embodiment of the invention is the use of a soluble biopolymer salt to form a fastening device by molding. The biopolymer powder is mixed or kneaded with water to a moisture level lower than what is needed to make a flowing solution of the biopolymer in water. The formed paste can then be shaped to the desired shape using a mold. Finally, the device may optionally be dried. Upon drying, the shape will be maintained, although the dimensions of the device might be altered due to shrinkage as water evaporates. This shrinkage can be controlled, i.e. by using a filler, controlled drying or by other means. The dried device is rigid with high strength, both tensile and torsional.

In a second embodiment, the biopolymer/water paste can be extruded through a nozzle to form plugs, bolts, anchors and pins, or other cylindrical shapes. The nozzle diameter and predicted shrinkage can give an implantable device with controlled thickness.

In a third embodiment of the invention relates to a device that is formed by mechanical means, such as, for example, milling. This can be done by forming a larger object of biopolymer and water, and after drying, mechanically shaping the object into the desired shape. This process should yield a device with controlled dimensions.

Another aspect of the invention includes filling a hollow screw, plug, bolt, anchor or pin made by one of the methods of the invention with a biopolymer based hydrogel. This hydrogel can contain osteoinductive materials, osteoconductive materials or tissue regenerative additives as for example growth factors, cell adhesion peptide sequences, osteoprogenitor cells, fibroblasts, cartilage, bone cells, including osteoblasts and osteoclasts, blood vessel cells, including vascular endothelial and perivascular endothelial cells, any genetically engineered cells that secrete therapeutic agents, such as proteins or hormones for treating disease or other conditions, genetically engineered cells that secrete diagnostic agents and stem cells. These materials can be used as a filler in devices of the present invention without incorporation into a hydrogel. The hydrogel can be manufactured by any method known in the art. Preferably the gel is set after or during filling the hollow device induced by for example temperature change or a self gelling alginate system as described by Melvik et al. (WO 2006/044342 A2), the disclosure of which is hereby incorporated by reference for the purpose of describing the self-gelling alginate.

With use of hollow or gel filled devices, the implant mass will be reduced and the surface area will be larger which may further increase the substitution rate of tissue. This may allow regeneration of tissue from both inside and outside of the device. If the tissue structure is created from the inside of the structure, the loss of mechanical strength of the device as it degrades may be less important.

The device of the present invention may optionally contain one or more biopolymer fibers. The fiber content of the device may range, for example, from about 5 to about 100% and, more preferably, fiber-containing devices will contain from about 30 to about 100% fiber. The fibers typically contain at least 85% solids. The biopolymer fibers can be prepared using any known technique. Also, a variety of different types of fibers may be prepared including, for example, non-crosslinked fibers, ionically crosslinked fibers or covalently crosslinked fibers. The degree of crosslinking can be stoichiometric or sub-stoichiometric, as desired to obtain the particular properties sought for a particular device or part of a device. In this manner, partial crosslinking can be employed as one method for providing a controlled rate of degradation of the fiber. Crosslinking can be carried out on either dry biopolymer material or wet biopolymer material. The rate of degradation or resorption of the biopolymer system may be controlled by varying the degree of cross-linking and the molecular weight of the components using any suitable technique, one illustrative technique being described in, for example, Kong, et al “Controlling rigidity and degradation of alginate hydrogels via molecular weight distribution,” Biomacromolecules, 2004, 5, 1720-1727, the disclosure of which is hereby incorporated herein by reference for a description of this technique.

The fibers may have a diameter, for example, in the range of 100 nm to 1 mm. During the manufacture of the fibers, any of the various materials described herein for incorporation in the device of the present invention may also be optionally included in the fibers.

The fibers used to manufacture the device can be of similar type in relation to diameter, biopolymer used, type of crosslinking and degree of crosslinking, or mixtures of different types of fibers, which vary in one or more of these properties, may also be used. Combinations of fibers from cationic and anionic biopolymer can be used to modify the stability of the device as ionic interactions will take place between the polymers and further stabilize the device. The fibers may be used as wet fibers to fabricate the device, prior to drying the wet fiber. In such case, wet fibers typically comprise from 0.1-15% by weight of biopolymer such as alginate, based on the total weight of the fiber.

The fibers may be incorporated in the device prior to application of pressure to form the device. Thus, the fibers may be molded into the device or co-extruded with other materials to form the device. In this manner, the fibers can be used to alter the properties of the device in the desired manner by, for example, altering the strength or degradation rate of the device.

The device of the present invention may also be modified by use of one or more treatments applied to the device at one or more stages of the fabrication process. For example, the device may be treated once with a biopolymer solution to provide a protective coating layer on the exterior of the device. Alternatively, the device may be treated after application of pressure and/or after being pre-shaped.

In one embodiment, the device may be treated in an aqueous bath comprising at least one of a degradable biopolymer, an uncrosslinked degradation controlling agent, an imaging agent, a gelling agent such as a gelling ion, an alcohol a tissue regenerative additive, a cell adhesion sequence or a pharmaceutically active agent selected from, but not limited to, a growth factor agent, an antiseptic, an anticoagulant, an antibiotic, an anti-inflammatory, a pain-killer, a chemotherapeutic agent, and an anti-infective agent, a protein or a drug to modify the properties of the device. In some embodiments, the device is treated in a solution of at least one gelling agent to gel the biopolymer and form a continuous, gelled layer. At least one gelling agent may be present in an amount of 0.01-10 weight percent of the aqueous bath. This treatment may be used in combination with one or more of the other treatments discussed above. The treatment(s) may last for up to 24 hours. This bath may also optionally include one or more biopolymers, non-crosslinked degradation control agents, imaging agents, pharmaceutically active agents, cell adhesion peptide sequences and growth factor agents, as desired. The growth factor agent used in the various methods of the present invention may be selected from bone morphogenic proteins, transforming growth factors (beta), fibroblast growth factors, platelet derived growth factors, vascular endothelial growth factors, insulin-like growth factors, epidermal growth factors and mixtures thereof.

Another embodiment of the invention includes treating the shaped device in an aqueous biopolymer solution. For example, if gelled alginate fibers are present in the device, a treatment in alginate solution will initiate dissolution of the alginate fibers as the gelling ions from the fibers will be shared with the surrounding alginate solution. An exemplary biopolymer solution may be a solution of sodium alginate. This will give a partly gelled alginate hydrogel surrounding the device, which, when dried, will form a film or a coating. Before drying, the device may be treated in an aqueous bath containing gelling ions to further add gelling ions to the coating layer in order to modify the degradation rate and/or swelling properties. The biopolymer solutions may optionally contain a plasticizer to reduce brittleness and modify hydration rates.

The film may, upon hydration after insertion, swell to fill potential voids between e.g. the bone and the inserted device, to interlock the device. The pressure caused by the swelling may also stimulate the healing of the injured tissue. The film can contain tissue regenerative agents as e.g. growth factors, antibiotics, peptide sequences or drugs. In general, film thickness can be controlled by the concentration of the biopolymer solution, viscosity of the biopolymer solution or the residence time the device is located in the biopolymer solution. When coating layers are added during manufacturing, layers containing different materials can be used to modify, for example, drug release and degradation properties. Such coatings may include, for example, sustained release agents, immediate release agents and delayed release agents. The coating layer may also contain any of the other agents discussed above for inclusion in the biopolymer, if desired. The coating layer is preferably applied on the exterior of the device.

The present invention is now described in more detail by reference to the following examples, but it should be understood that the invention is not construed as being limited thereto. Unless otherwise indicated herein, all parts, percents, ratios and the like are by weight.

### EXAMPLES

### Example 1

5.54 grams of alginate, LN-8 (Lessonia Nigrescens alginate), was mixed with 6.2 grams deionized water in a mortar until a uniform rubber-like paste was formed. This paste had a calculated moisture content of 54%. Some of the mixing was done by hand due to the very high viscosity of the mixture. When the paste appeared homogeneous under visual inspection, part of the mass was molded into a screw-like shape using a nut. The nut was packed/filled with the alginate mass as compact as possible and left for drying at 25° C. and 35% RH. After drying, the device shrank to a volume that permitted the device to be withdrawn from the mold without rotating it. The threads appeared the same as regular threads on a screw.

### Example 2

5.9 grams alginate (LN-8) and 7.6 grams water was mixed in similar manner as in Example 1, and the resulting paste had a calculated moisture level of 61%. The paste was then extruded through a 9 millimeter nozzle to form pins. The pins were left for drying on bench at 25° C. and 35% relative humidity. After drying, the pins had a diameter of 6.58 millimeters, and a dry matter content of 94.2%. One pin was measured on a SMS Texture Analyzer, TA-XTi, applying two different methods and probes.

First, a guillotine probe was used, where a sharp axe-like probe compresses the sample towards a slit of 3.2 millimeters oriented transversely to the pin. In this test, the pin survived the maximum load, which was 40 kilograms.

In the second test, the pin was attached between two probes, each with a clamp, fastening the pin in a vertical direction. The instrument then measured force in tension of the sample before it breaks. Again, no breakage was seen at the maximum tension force, which was 40 kg.

### Example 3

This example demonstrates that shrinkage of extruded biopolymer pastes varies depending on the paste formulation.

Eight different formulations were tested with variations in the type of raw material employed, including blends of different raw materials. The amount of water added was kept to a minimum ensuring a homogeneous paste. The extruded materials were made by first mixing the biopolymer powders, if more than one biopolymer powder was used, and then water was added and a uniform paste was made using a mortar and pestle. The paste was then filled into a plastic syringe (20 milliliters), and force was applied by hand to compress the material before the paste was extruded through a 7.5 millimeter diameter outlet. The extruded plugs were dried, uncovered, at ambient temperature for three days.

The different formulations and the diameters of the dried material are presented in Table I. The alginates and chitosans, named PRONOVA and PROTASAN, respectively, are available from NovaMatrix, Sandvika, Norway. The hyaluronate (SODIUM HYALURONATE PHARMA GRADE 80) is available from Kibun, Tokyo, Japan. PRONOVA UP VLVG and PRONOVA UP MVG represent sodium alginates with very low viscosity (VLVG has a viscosity <20 mPas) and medium viscosity respectively (MVG has a viscosity >200 mPas). PRONOVA UP CAM is a calcium alginate with a G/M ratio<1. PROTASAN UP CL 213 is a chitosan chloride salt with a viscosity of 20-200 mPas and a degree of deacetylation of 70-90%.

TABLE I
Formulation and diameter of dried extruded biopolymer plugs.

| | Amount | Amount | Diameter, |
|---|---|---|---|
| Polymer | polymer, [g] | water, [g] | [mm] |
| PRONOVA UP VLVG | 4 | 5 | 5.5 |
| PRONOVA UP MVG | 4 | 5 | 5.1 |
| PRONOVA UP MVG: | 2/2 | 7 | 5.4 |
| PRONOVA UP CA M (1:1) | | | |
| PROTASAN UP CL 213 | 4 | 9 | 4.8 |
| Hyaluronate | 4 | 4 | ~7 |
| PROTASAN UP CL 213: | 2:2 | 6 | 5.3 |
| PRONOVA UP MVG (1:1) | | | |
| PROTASAN UP CL 213: | 2:2 | 5 | 6.4 |
| Hyaluronate (1:1) | | | |
| PRONOVA UP MVG: | 2:2 | 5 | 5.4 |
| Hyaluronate (1:1) | | | |

The data show that the materials comprising hyaluronate will shrink less than the other materials. This may indicate that the hyaluronate-containing materials are more hygroscopic than the other biopolymers that were tested.

### Example 4

This example shows that as an extruded biopolymer plug hydrates in a model physiological solution, a highly viscous layer is created around the plug. The biopolymers in this layer will interact with surrounding fluids, materials and cells. This example also shows that the inner core retains its strength even if hydration is initiated. This may be beneficial since bone forming cells can be mobile in this layer, thereby moving inwardly in the device.

A Ringer solution was made according to the US Pharmacopeia (USP23) from the following salts: 6.02 grams NaCl, 0.21 grams KCl and 0.231 grams CaCl*2H₂O dissolved in 700 milliliters of deionized water. For some of the formulations, three or four plugs (each with a length of about 2 centimeters) of the same material were put in a weighing boat (125 milliliters) containing 75 milliliters of Ringer solution. The samples were kept in this solution at ambient temperature for two hours before the solution was decanted, and the dimensions were measured. Then the strength of the partly rehydrated materials was tested with a texture analyzer (Model TA-XT2) using a rounded blade/guillotine test fixture. The blade speed was 0.5 millimeters per second. Most of the samples showed a biphasic strength curve indicating the forces required to break both the outer (gelled) shell and the inner core.

This example also demonstrated that the materials of the device will swell upon hydration, and that the swelling and surface properties of the materials can be influenced by the specific method of forming the material.

### Example 5

This example shows the manufacture of extruded biopolymer plugs containing biopolymer fibers.

Plugs were made as described in Example 3 from deionized water and PRONOVA UP MVG except that 5% by weight of alginate fibers of 1 centimeter in length were added to the alginate powder before the water was added and the paste was made. When extruded, the fibers were visible, entangled in the plug. The samples were dried for three days, uncovered, at ambient temperature. The dried samples were tested using a three point bending test with use of the texture analyzer (TA-XT2). The speed was 0.5 millimeter per second and the gap between the two bars was 1″. There was no significant difference in the strength measured for plugs containing fibers compared with plugs without fibers in this experiment.

The same formulations were also rehydrated for two hours and then the strength was measured with the texture analyzer and the rounded blade/guillotine test fixture as described in Example 4, except a Hanks' Balanced Salt Solution (H8264, SigmaAldrich Chemie GmbH, Steinheim, Germany) was used as a model physiological solution. Differences were visible between the fiber-containing samples and the samples which did not contain fiber. The fiber-containing samples showed an amorphous coating around the extruded core but within the gel coating, and a swelled hydrated layer surrounding the sample. The coating and swelled hydrated layer were absent in the samples which did not contain fiber.

### Example 6

6.40 g of hyaluronate (SODIUM HYALURONATE PHARMAGRADE 80, Kibun Food Chemifa Co., Ltd, Tokyo, Japan, dry material content (DMC)=93.5%) was mixed with 8.62 g deionized water in a mortar until a uniform rubber-like paste was formed. This paste had a calculated moisture content of 60%. Some of the mixing was done by hand due to the high viscosity of the paste and mixing was continued until the paste was visually homogeneous. A portion of the paste was packed/filled into a metal tube with a length of 40 millimeters and an inner diameter of 6 millimeters. A close fitting metal plunger was put into one end of the metal tube and then pushed using maximum hand compression and held for 15 seconds against a flat surface on the laboratory bench. The compression step was repeated. After the compression step, the plunger was pressed to allow removal of the bolt from the metal tube and the bolt was dried for two days at ambient conditions on the laboratory bench. The diameters of the dried bolts were 4.3 millimeters +/−0.2 millimeters.

The strength of the dried hyaluronate bolts was measured on a SMS Texture Analyzer, TA-XT2 with a 25 kg load cell, using a HDP/3PB Three Point Bend Rig with a base gap of 10 millimeters. The mode selected was: “measure force in compression” with a pre-test speed of 0.5 millimeters per second and a test speed of 0.2 millimeters per second. The distance was 10 millimeters and the trigger force was set to 5 grams. The force was applied normal to the major axis of the bolt. No breakage was seen for three out of four bolts at maximum compression force, which was 40,000 grams. The force applied to the bolt that broke was 35,000 grams.

As model for physiological solution, a Hanks' balanced salt solution was used. Four or five extruded bolts were placed in a 100 milliliter weighing boat containing 75 milliliters of Hanks' balanced salt solution. The bolts were fully covered by the Hanks' solution. The samples were kept in this solution at room temperature for two hours. The strength of the rehydrated materials was tested with a SMS Texture Analyzer, TA-XT21 with a 5 kilogram load cell, using a HDP/BSG Blade Set with Guillotine as the probe. The force was applied normal to the major axis of the bolt. The mode selected was: “Measure force in compression” with pre-test speed 0.5 millimeters per second and a test speed of 0.25 millimeters per second. The distance was 10 millimeters and the trigger force was set to 1 gram. The force that had to be applied to break the rehydrated bolts was 3900 grams±700 grams (n=3).

### Example 7

Seven different formulations were prepared using the preparation and testing methods in Example 6 for the following biopolymers: PRONOVA UP VLVG alginate (F_{G}=0.69, DMC, 93.9%, viscosity=5 mPas); PRONOVA UP MVG alginate (F_{G}=0.72, DMC=89.3%, viscosity=572 mPas); PROTASAN UP CL 110 chitosan (DMC=91.4%, viscosity=12 mPas) and Sodium Hyaluronate Pharmagrade 80 (DMC=93.5%).

The pastes were prepared at a calculated moisture content of 60% except for the chitosan paste which had a calculated moisture content of 76%. In the formulations containing two biopolymers, the dry powders were premixed before MilliQ water was added. At the moisture content tested, the paste prepared from a 1:1 mixture of hyaluronate and chitosan was soft, very elastic and stretchable compared to the pastes prepared from only chitosan or alginate (MVG) which felt rougher and drier than the paste prepared from a 1:1 mixture of hyaluronate and chitosan. The pastes made out of only hyaluronate or alginate (VLVG) were very soft, but not as elastic and stretchable as the paste made out of the 1:1 mixture of hyaluronate and chitosan.

Bolts were prepared and dried as in Example 6. Dried bolts each having the same composition were rehydrated together in Hanks' solution. After testing the bolts which had been placed in Hanks' solution for 2 hours, the failed bolts were examined Some compositions were observed to have a transparent, highly viscous layer covering the bolt while retaining a non-transparent core. This structure was not observed for dried bolts prepared with the mixtures of hyaluronate and chitosan or alginate mixed with chitosan, respectively, after 2 hours in Hanks' solution

The results of the strength measurements of the bolts made from different biopolymers as well as the results for the bolts made from mixtures of biopolymers, both dry and after hydration for 2 hours, are shown in Table II.

TABLE II
Mechanical properties of average force (n = 3-5 ±
SD) and maximum force to break the bolts prepared from
biopolymers and biopolymer blends

| | Dry bolt | | Rehydrated bolt | |
|---|---|---|---|---|
| | | Maximum | | Maximum |
| Polymer | Force, [g] | force, [g] | Force, [g] | force, [g] |
| Alginate (VLVG) | 34900 ± 1900 | 37100 | 3500 ± 1600 | 5300 |
| Alginate (MVG) | 16200 ± 1700 | 18200 | 2100 ± 200 | 2200 |
| Chitosan | 9600 ± 1600 | 11400 | 1600 ± 60 | 1700 |
| Hyaluronate | 35000* | >40000 | 3900 ± 1200 | 4300 |
| Alginate (MVG): | 22000 ± 3100 | 24000 | 2100 ± 600 | 2800 |
| chitosan (1:1) | | | | |
| Chitosan: | 33300* | >40000 | 5100 ± 400 | 5300 |
| Hyaluronate (1:1) | | | | |
| Alginate (MVG): | 18800 ± 1400 | 20900 | 500 ± 100 | 600 |
| Hyaluronate (1:1) | | | | |

*Only one bolt cracked during measurement. The force given is the value for this bolt.

The data from Table II show that the strongest dried bolts are hyaluronate only or hyaluronate in combination with chitosan, followed by the VLVG alginate bolts. One out of five bolts made of pure hyaluronate or a 1:1 mixture of chitosan and hyaluronate, respectively, cracked during the strength measurement. The remaining four were sufficiently strong that 40 kilograms of pressure were not enough to break them. In the rehydrated condition (after 2 hours of rehydration), bolts made of a 1:1 mixture of chitosan and hyaluronate were the strongest followed by bolts made from VLVG alginate and from hyaluronate. In the dry condition bolts made of chitosan were the weakest, while bolts made of a 1:1 mixture of MVG alginate: hyaluronate were the weakest in the wet condition.

### Example 8

Pastes made from hyaluronate only or from a 1:1 mixture of chitosan and hyaluronate were prepared as in Example 6. The chitosan and hyaluronate were premixed in dry condition before MilliQ water was added. The resulting paste had a calculated moisture content of 60%. The bolts were prepared using the metal tube except that a 2-3 millimeter thick plug of non-swellable, non water-absorbable rubber was placed in each end of the metal tube to ensure that the paste was retained within the tube during compression. A metal plunger 5.8 millimeters in diameter was inserted into one end of the tube against the rubber plug and pushed in compression for 5 minutes using a vise. The bolts were then either air-dried on the bench or freeze dried for 24 hours using a Heto Hetosicc CD 2.5 freeze dryer.

The strength of the dried hyaluronate bolt was measured as described in Example 6 except that the gap on the HDP/3PB Three Point Bend Rig was increased from 10 millimeters to 15 millimeters. The strength of the rehydrated hyaluronate bolts was measured as described in Example 6. The length and diameter of the bolts were measured using a caliper, in a dry condition and in a rehydrated condition after 2 hours in Hanks' solution. The degree of swelling of the rehydrated bolts was calculated as the difference between the rehydrated diameter and the dry diameter divided by the dry diameter. The results are presented in Table III.

TABLE III
Results from size and breakage strength measurements of air dried
and freeze dried bolts before and after rehydration in Hanks' balanced salt
solution
(n = 3-5, ±SD).

| | Dry bolt | | | Rehydrated bolt | | | |
|---|---|---|---|---|---|---|---|
| | Force | Maximum | Diameter | Force | Maximum | Diameter | Swelling |
| Polymer | [g] | force [g] | [mm] | [g] | force [g] | [mm] | [%] |
| Hyaluronate | 32500 ± 2200 | >40000 | 4.1 ± 0.1 | >6400 | >6400 | 8.9 ± 0.8 | 115 ± 20 |
| Air dried | | | | | | | |
| Hyaluronate | 35300 ± 1800 | 37000 | 5.0 ± 0.2 | 100 ± 20 | 140 | 9 ± 0.4 | 80 ± 14 |
| Freeze | | | | | | | |
| dried | | | | | | | |
| Chitosan:Hyaluronate | 24600 ± 3600 | >40000 | 4.2 ± 0.2 | 3700 ± 2100 | >6400 | 5.8 ± 0.7 | 42 ± 3 |
| (1:1) | | | | | | | |
| Air dried | | | | | | | |
| Chitosan:Hyaluronate | 15500 ± 2000 | 27000 | 4.9 ± 0.2 | 2100 ± 1300 | 3500 | 6.1 ± 0.5 | 25 ± 13 |
| (1:1) | | | | | | | |
| Freeze | | | | | | | |
| dried | | | | | | | |

The results of this study show that air dried bolts were stronger than freeze dried bolts, both in the rehydrated and dry states, particularly when based on the maximum force that had to be applied to break the bolt. The difference in breakage strength was also significant for the rehydrated bolts made from only hyaluronate. The bolt made of hyaluronate as the only biopolymer was more rehydrated than the bolt made from a mixture of hyaluronate and chitosan. The 1:1 chitosan:hyaluronate bolt had a larger inner core of dry material after 2 hours in Hanks' balanced salt solution than the bolt made of pure hyaluronate in which the inner core appeared to be partially rehydrated.

As shown in Table III, freeze dried bolts swell less than bolts that have been air dried. By mixing hyaluronate with chitosan, the swelling of the bolt was reduced by approximately 60% compared to bolts made out of hyaluronate as the only polymer. Table III also shows that freeze dried bolts had less shrinkage in the radial direction than the corresponding air dried bolts.

### Example 9

Bolts made of hyaluronate and chitosan were made with the following ratios 1:3, 1:1 and 3:1, of hyaluronate:chitosan on a solids basis. The strength and size of the bolts were measured in a similar manner as described in Example 8. The chitosan and hyaluronate were premixed in dry condition before MilliQ water was added. The resulting paste had a calculated moisture content of 60%. The results are presented in Table IV.

TABLE IV
Results from breakage strength and size measurements of
hyaluronate:chitosan bolts mixed in different ratios (n = 3-5, ±SD).

| | Dry bolt | | | Rehydrated bolt | | | Swelling |
|---|---|---|---|---|---|---|---|
| | Force, | Maximum | Diameter, | Force, | Maximum | Diameter, | Average |
| Polymer | [g] | force, [g] | [mm] | [g] | force, [g] | [mm] | [%] |
| Hyaluronate:Chitosan | 6400 ± 1600 | 8800 | 4.2 ± 0.2 | 460 ± 210 | 690 | 7.6 ± 0.7 | 73 ± 9 |
| (1:3) | | | | | | | |
| Hyaluronate:Chitosan: | 24600 ± 3600 | >40000 | 4.2 ± 0.2 | 3700 ± 2100 | >6400 | 5.8 ± 0.7 | 42 ± 3 |
| (1:1) | | | | | | | |
| Hyaluronate:Chitosan: | 22900 ± 1900 | 25600 | 4.2 ± 0.1 | 4300 ± 950 | 5600 | 7.1 ± 0.5 | 70 ± 13 |
| (3:1) | | | | | | | |

Table IV shows that mixing hyaluronate with chitosan in different ratios gave extruded bolts with different strengths and sizes. A bolt with excess chitosan was determined to be a much weaker bolt than a bolt with equal amounts of hyaluronate and chitosan. From the average values presented in Table IV, hyaluronate:chitosan bolts made in 1:1 and 3:1 ratios seem to have similar strength properties. However, based on the maximum force values, it appears that equal amounts of hyaluronate and chitosan produced the strongest bolts since some of the bolts did not break during measurement.

Table IV also shows that the mixtures of hyaluronate and chitosan in 1:3 and 3:1 ratios swelled the most. The formulation that had the highest break strength was also the formulation that exhibited the least swelling during hydration.

### Example 10

Bolts were prepared as described in Example 8 using a paste with a calculated moisture content of 64% prepared with MilliQ water and alginate (MVG). These bolts were dried and 6 bolts were placed in a 2% solution of chitosan for 30 minutes and then removed from the solution and air-dried for two days. The strengths of the coated and uncoated bolts were measured in a similar manner as described in Example 8. The results are presented in Table V.

TABLE V
Results from breakage strength measurements of alginate bolts,
and chitosan coated alginate bolts. (n = 3-5, ±SD)

| | Dry bolt | | Rehydrated bolt | |
|---|---|---|---|---|
| | | Maximum | | Maximum |
| Polymer | Force, [g] | force, [g] | Force, [g] | force, [g] |
| Alginate (MVG) | 10100 ± 3100 | 13000 | 1500 ± 800 | 2200 |
| Coated alginate | 15300 ± 6700 | 22300 | 190 ± 70 | 290 |
| (MVG) | | | | |

This experiment shows that dry chitosan coated alginate bolts made by this process were stronger than uncoated bolts, while rehydrated coated bolts were weaker than uncoated bolts.

### Example 11

Bolts of hyaluronate and chitosan in 1:1 ratio and bolts of hyaluronate and chitosan in 1:1 ratio with 10% added NaCl, were made and measured in a similar matter as described in Example 3. The chitosan and hyaluronate were premixed in dry condition before deionized water was added. The resulting paste had a calculated moisture content of 60%. Results from the strength measurements are presented in Table VI.

TABLE VI
Results from strength measurements of 1:1 hyaluronate:chitosan bolts
with or without 10% NaCl added. (n = 3-5, ±SD)

| | Dry bolt | | Rehydrated bolt | |
|---|---|---|---|---|
| | | Maximum | | Maximum |
| Polymer | Force, [g] | force, [g] | Force, [g] | force, [g] |
| Hyaluronate: | 24600 ± 3600 | >40000 | 3700 ± 2100 | >6400 |
| chitosan (1:1) | | | | |
| Hyaluronate: | 20100 ± 4500 | 25600 | 2000 ± 600 | 2900 |
| chitosan (1:1) + | | | | |
| 10% NaCl | | | | |

Table VI shows that adding NaCl to the mixture produces bolts that are weaker in strength than the same formulation without NaCl. Without being bound by theory, the added ions might shield the charges of the polymers and therefore prevent interaction between hyaluronate and chitosan and give weaker bolts.

### Example 12

Bolts of made of hyaluronate mixed with alginate (MVG) in 1:1 ratio, and bolts containing hyaluronate mixed with chitosan (CL 214) in 1:1 ratio were made in a similar matter as described in Example 3. The biopolymers were premixed in a dry condition before deionized water was added. The resulting paste had a calculated moisture content of 60%. Results from the strength measurements are presented in Table VII.

TABLE VII
The effect on breakage strength and swelling on bolts
by hydration for 2, 4 and 8 hours in Hanks' balanced
salt solution. (n = 3-5, ±SD)

| | Hydration | | |
|---|---|---|---|
| | time, | Swelling, | |
| Formulation | [hours] | [%] | Force, [g] |
| Alginate (MVG): | 2 | 127 ± 8 | 1200 ± 600 |
| Hyaluronate (1:1) | | | |
| Alginate (MVG): | 4 | 177 ± 9 | 50 ± 5 |
| Hyaluronate (1:1) | | | |
| Alginate (MVG): | 8 | 186 ± 11 | 20 ± 6 |
| Hyaluronate (1:1) | | | |
| Chitosan: | 2 | 42 ± 3 | 3700 ± 2100 |
| Hyaluronate (1:1) | | | |
| Chitosan: | 4 | 62 ± 6 | 250 ± 70 |
| Hyaluronate (1:1) | | | |
| Chitosan: | 8 | 64 ± 13 | 120 ± 20 |
| Hyaluronate (1:1) | | | |

Table VII shows that swelling of the bolts increased over time. Most swelling occurred during the first 2 to 4 hours, while there was no increase in diameter from 4 to 8 hours. The strength of the bolts decreased rapidly from 2 to 4 hours. There was less of a strength decrease from 4 to 8 hours.

The swelling and the strength of the bolt can be controlled by varying the biopolymer that is mixed with hyaluronate. Mixing hyaluronate with chitosan gives bolts that have higher breakage strength and less swelling than bolts made by mixing hyaluronate with alginate.

### Example 13

Alginate bolts were made by first hand kneading alginate pastes comprising 50% or 65% water and 50% or 35% alginate powder, respectively (where the water content in the paste is the total amount of water added together with water present in the alginate powder). The paste was kept in the refrigerator overnight to obtain a more hydrated/uniform paste. The alginate paste was then fed into a Brabender extruder. The diameter of the extruder screw was 20 millimeters and the length of the extruder screw was 50 centimeters. The rotation speed was approximately 10 rpm and the nozzle diameter was either 0.75 millimeter or 1.00 millimeter. Alginate extrudates were collected in suitable containers from the nozzle of the extruder for drying, e.g. petri dishes.

When the extruded alginate pastes were freeze dried, it was found to be important to keep the frozen bolts frozen during sublimation in the freeze drier to avoid formation of mobile water in a layer around the polymer. When the water partly melted a collapse of the structure was seen resulting in more shrinkage of the bolt. Optimized freeze drying of a wet alginate bolt comprising 65% water extruded from a nozzle with a diameter of 0.75 millimeter gave a dry bolt with a diameter of 0.65-0.70 millimeter. About 92% dry matter content was obtained after 3 to 4 hours of freeze drying.

When the alginate pastes were air dried the humidity in the surrounding air was controlled to reduce cracks and roughness. As the surface of a bolt dries first this will result in a moist core. As the water from the core evaporates through the outer shell this may cause cracking. This problem was solved by introducing a humidity gradient between the wet bolt and surrounding air to reduce evaporation from the surface of the bolt and allow diffusion of the water from center of the bolt to the surface before it is completely dry. This humidity gradient was created by drying the bolts in a plastic bag with a small opening. An air dried alginate bolt made from paste comprising 65 to 75% water shrinks 25 to 30% in the radial direction and somewhat less in the axial direction as a result of drying. About 92% dry matter content was obtained after 3 to 4 days of drying.

The alginate density of air dried materials was found not to be dependent on the water content or chemical composition of the alginate in the paste. The freeze dried materials gave increased water content in the paste, a decreased alginate density and they became more porous.

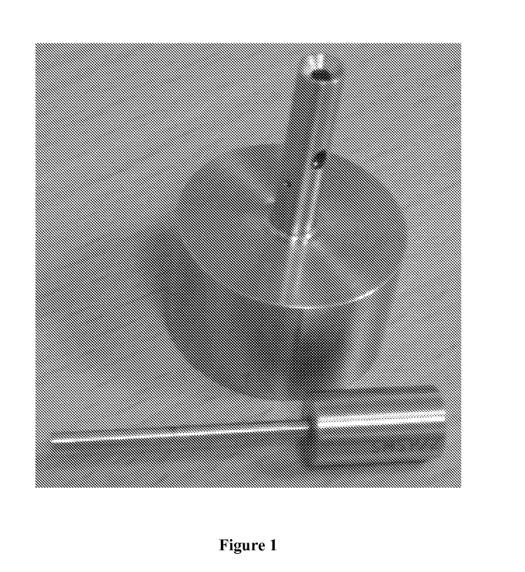

The breaking strength and breaking time was measured with use of a Texture Analyzer from Stable Micro Systems (TA-XT21). The breaking strength was measured as the force required to break a bolt, where the bolt was fixed in a test jig as presented in FIG. 1. Force was applied normal to the major axis of the bolt until breakage occurred. The mode selected was: “Measure force in compression” and pre-test speed and test speed were 1.0 millimeters per second and 0.1 millimeters per second, respectively. The distance the probe traveled was 1.2 millimeters and the trigger force was set to 30 grams. The breaking time was measured by first adding 0.2 milliliters of MilliQ-water (at a temperature 21 to 23° C.) to the bolt. Subsequently, a constant force of 200 grams was applied while the probe moved at 0.1 millimeter per second and the time when breakage occurred was determined. The pre-test speed was set to 2.0 millimeters per second and the trigger force was set to 30 grams. The measurement started about 15 to 20 seconds after the bolt was rehydrated.

Shown in the front of FIG. 1 is the standard test probe SMS P/2 (diameter: 2 millimeter). Also shown in FIG. 1 is a specially designed jig. The bolt is placed vertically across the neck (outer diameter: 4.8 millimeter, inner diameter: 2 millimeter) of the jig between two holes of 1 millimeter in diameter. Just above where the bolt is placed is a hole for injection of water. The probe is fastened to the texture analyzer and moves down into the neck and measurement starts as it meets the fixed bolt.

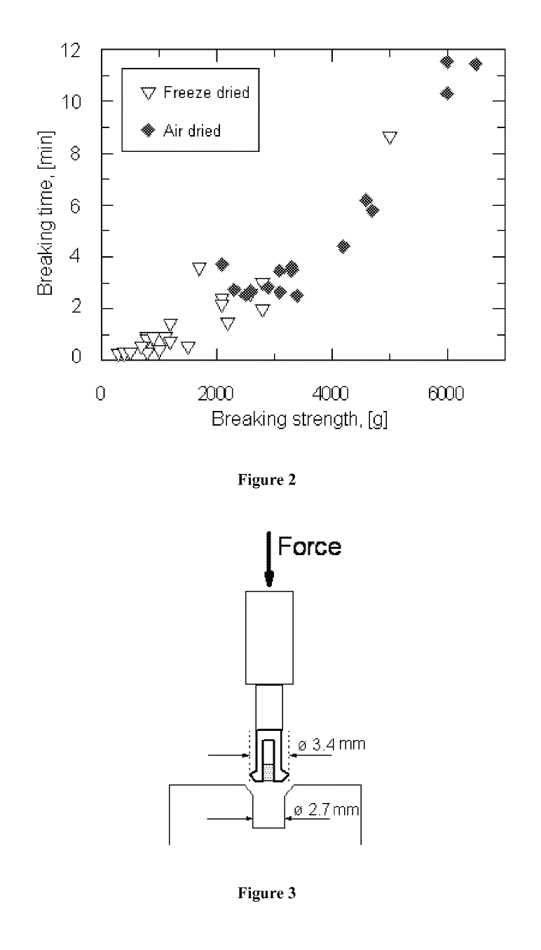

FIG. 2 presents breaking time as a function of breaking strength comparing freeze dried and air dried alginate bolts. The data show that dry freeze dried bolts are weaker and break faster after hydration as compared to air dried alginate bolts.

### Example 14

The preparation of freeze dried alginate bolts and breaking strength measurements were performed as described in Example 13. The alginate materials, their chemical composition, Brookfield viscosity (measured at 1% solids in water, 20° C.) and the resulting average breaking strength are presented in Table VIII. The extruder nozzle diameter and amount of water added during preparation of the paste was constant.

TABLE VIII
Alginate characteristics and resulting breaking
strength of a freeze dried alginate bolt.

| | | Viscosity 1% | Breaking |
|---|---|---|---|
| Alginate | F_{G} | solution, [mPas] | strength, [g] |
| PRONOVA LVG | 0.7 | 5 | 400 |
| PROVOVA LVG | 0.7 | 25 | 800 |
| PRONOVA LVG | 0.7 | 190 | 1200 |
| PRONOVA LVM | 0.4 | 5 | 1100 |
| PRONOVA LVM | 0.4 | 196 | 2100 |

The table shows that an increase in molecular weight of the alginates used for bolt preparation increased the resulting breaking strengths of the bolts. Additionally, a decrease in the fraction of guluronate moieties (F_{G}) also increased the breaking strength of the bolts.

### Example 15

Both freeze dried and air dried alginate bolts were prepared as described in Example 13, with a constant nozzle diameter used for all pastes. The different alginates used were PRONOVA LVM (196 mPas, F_{G}: 0.4) and PRONOVA LVG (190 mPas, F_{G}: 0.7). Table IX presents test results for alginate bolts made from alginates with different chemical compositions, pastes with varying water content and bolts dried using different methods.

TABLE IX
Relation between average breaking strength and water content
in the alginate pastes used for bolt preparation.

| | | Breaking strength, [g] | | |
|---|---|---|---|---|
| | Drying | Water content | | |
| Alginate | Method | 35% | 65% | 75% |
| PRONOVA LVM | Air dried | 4600 | 4200 | — |
| PRONOVA LVM | Freeze dried | 5000 | 2800 | 1500 |
| PRONOVA LVG | Air dried | — | 2900 | 2100 |
| PRONOVA LVG | Freeze dried | — | 1200 | 1200 |

The results in Table IX indicate that increasing the water content in the paste will decrease the breaking strength of the dried alginate bolt.

SEM pictures of different alginate bolts were prepared by first placing a dried alginate bolt into liquid nitrogen (N₂), breaking the bolt manually to ensure a clean surface fracture, coating with a thin layer of gold and then fixing in the microscope. The pictures of the bolts made from freeze dried PRONOVA LVG pastes comprising 65% and 75% water did not have visually observable differences in pore size or pore structure. This observation corresponds well with the breaking strength results in Table IX which indicate no difference between the two samples. The pictures of alginate bolts made from freeze dried PRONOVA LVM pastes comprising 63% and 33% water show a more compact structure for the bolt made from the paste comprising 33% water. Only a few pores are visible and it appears that not all particles are dissolved for the bolt made from the paste comprising 33% water. The compact structure may also explain the high measured breaking strength.

### Example 16

Increased breaking strength is achieved by increasing bolt and nozzle diameter. By increasing the nozzle diameter from 0.75 millimeter to 1.00 millimeter, the breaking strength increases by a factor 1.5-2. The diameter of the dried bolt increases by a factor 1.25, which is proportional to the nozzle diameter. These changes will be the same for freeze dried and air dried bolts and also alginates rich in either mannuronate or guluronate. A linear relation was found between breakage time and bolt diameters from 0.4 millimeter to 0.7 millimeter.

### Example 17

Freeze dried and air dried alginate bolts were made as described in Example 13. The alginate used was PRONOVA LVM with viscosity in 1% solution of 144 mPas and F_{G}: 0.4. The paste comprised 65% water. Molecular weights (M_{W}) of the alginates from bolts before and after gamma irradiation at 32 kGy were determined by Size Exclusion Chromatography and Multi Angle Laser Light Spectroscopy (SEC-MALLS). The breakage strengths before and after sterilization were measured as described in Example 14, except that the pre-test and test speeds were 2.0 millimeters per second and 0.02 millimeters per second, respectively. The geometry of the bolt was also different as this time the normal compression was measured. The feet on the fork that keeps the bolt in place rest on the base and the force required to press the fork into the cylinder by breaking the bolt is read. The new test jig used to measure breakage strength of the bolt is presented in FIG. 3. The bolt is presented as depicted in FIG. 3. Table X presents the molecular weights and breakage strengths before and after gamma irradiation.

TABLE X
Molecular weight and breaking strength of alginate bolts before and after
gamma irradiation (n = 3, ±standard deviation of mean).

| | | | | Breaking |
|---|---|---|---|---|
| | Diameter, | Gamma | M_{w}, | strength, |
| Alginate bolt | [mm] | irradiated | [g/mole] | [g] |
| Freeze dried | 1.4 | No | 154 000 | 5500 ± 300 |
| Freeze dried | 1.4 | Yes | 56 000 | 5000 ± 150 |
| Air dried | 1.4 | No | — | 5600 ± 50 |
| Air dried | 1.4 | Yes | 58 000 | 6200 ± 100 |

The results presented in Table X show that the alginate was degraded during gamma irradiation, but the strengths of the dry alginate bolts were not affected by the treatment.

### Example 18

Bolts were made as described in Example 13, except that the nozzle diameter was either 1.5 millimeter for samples to be freeze dried, or 2.0 millimeter for samples to be air dried to thereby provide similar diameters for the dried bolts obtained from each drying process.

Table XI presents the chemical compositions and molecular weights of the different alginates used, the composition of the pastes, the drying method and the dimensions of the bolts.

TABLE XI
Alginate bolts.

| | Water | | Diameter | | | | |
|---|---|---|---|---|---|---|---|
| | content | | of dried | | | | Viscosity |
| | paste, | Drying | bolt, | | | | 1% sol., |
| ID | [%] | method | [mm] | Alginate source | Trade name** | F_{G} | [mPas] |
| 16.09A | 50 | Air | 1.5 | Ascophyllum | PRONOVA | 0.4 | 5* |
| | | dried | | nodosum | LVM | | |
| 16.09B | 50 | Freeze | 1.3 | Ascophyllum | PRONOVA | 0.4 | 5* |
| | | dried | | nodosum | LVM | | |
| 16.09C | 60 | Freeze | 1.4 | Laminaria | PRONOVA | 0.7 | 188 |
| | | dried | | hyperborean, stem | LVG | | |
| 16.09D | 60 | Air | 1.4 | Laminaria | PRONOVA | 0.7 | 188 |
| | | dried | | hyperborean, stem | LVG | | |
| 12.10A | 65 | Freeze | 1.4 | Lessonia | PROTANAL | 0.4 | 870 |
| | | dried | | nigrescens | HF 120 L | | |
| 12.10C | 65 | Air | 1.4 | Lessonia | KELTONE ® | 0.4 | 390 |
| | | dried | | nigrescens | HVCR | | |

*Thermal degradation of the alginate powder.
**The purity of the raw materials in terms of content of endotoxins is reported as filtered through 0.2 micron filter for the PRONOVA samples and 116 000 EU/g and 59 700 EU/g for PROTANAL and KELTONE ®, respectively. PROTANAL supplied from FMC Corp. Philadelphia, PA, USA and KELTONE ® from ISP Alginates, San Diego, CA, USA.

The irritation potential and local tolerance of the alginate bolts on rabbit muscle tissue following implantation was evaluated after exposure periods of 7 and 21 days. The bolts from Table XI with a length of 10 millimeters were sterilized by gamma irradiation (32 kGy) before implantation. An indwelling catheter (PhysioCath™, Data Sciences International, St Paul, Minn., USA) made of a polyurethane material was used as a control. The six alginate bolts and the polyurethane control bolts were intramuscularly implanted into the vertebral region into each of six New Zealand White rabbits. Three of the animals were sacrificed after 7 days and the remaining three rabbits after 21 days. Clinical observations, body weights, necroscopy—and histological findings, and tissue ingrowth were recorded. No clinical signs related to systemic toxicity were noted. Generally, there was a slight body weight loss following surgery, but overall body weight was considered to be unaffected by the treatment. The individual necroscopy—and histological findings are presented in Table XII.

TABLE XII
Individual necroscopy findings and histological findings

| ID | Necroscopy findings | Histology findings |
|---|---|---|
| 16.09A | Subcutaneous or muscle reddening was | 7 days: |
| | noted in two of the six animals (1 after | For the alginate bolt 12.10A, 1 |
| | 7 days, 1 after 21). A pale focus was | out of 3 of the animals had |
| | noted in the muscle of another animal | minimal fibrosis and mild to |
| | (21 days). Otherwise no abnormalities | moderate degeneration was |
| | were detected (NAD). | seen for all 3 animals. |
| 16.09B | A subcutaneous gelatinous texture was | All animals with implanted |
| | noted in one animal and a subcutaneous | bolts 16.09A, 16.09B, 16.09C, |
| | reddening was noted in another animal | 16.09D and 12.10C had |
| | after 7 days. A pale focus in muscle | minimal fibrosis and mild to |
| | was seen for all animals (7 days). | moderate degeneration. |
| | Subcutaneous reddening was seen for | For all animals a mild to |
| | one animal, subcutaneous thickening | moderate, mixed acute |
| | for another and a red focus in the | inflammatory response |
| | muscle of the third animal was noted | including macrophages, |
| | after 21 days. | neutrophils, lymphocytes and |
| 16.09C | A reddened lesion with pale centre was | plasma cells was noted. There |
| | seen in the muscle of one animal after 7 | was minimal to mild necrosis. |
| | days. The second animal had | 21 days: |
| | subcutaneous reddening and a pale | Minimal necrosis was found in: |
| | focus in the muscle, whereas the third | one of the three animals for |
| | animal had a subcutaneous pale focus. | bolts 16.09A and 16.09C. |
| | Muscle reddening was noted in one | two of the three animals for |
| | animal after 21 days. The second | bolts 16.09D and 12.10A. |
| | animal had pale focus in the muscle | Minimal to mild necrosis was |
| | whereas NAD was detected for the | seen on two of three animals for |
| | third. | bolts 12.10C. |
| 16.09D | Subcutaneous gelatinous texture was | No necrosis was seen for bolts |
| | noted for one animal after 7 days. The | 16.09B. |
| | second had muscle (subcutaneous) pale | For bolts 12.10C a slightly |
| | focus and a reddened centre, whereas | more severe prolonged reaction |
| | the third had a subcutaneous pale focus | was observed. For this sample |
| | and muscle reddening. NAD were | mild to moderate fibrosis and |
| | detected for all animals sacrificed after | moderate degeneration was |
| | 21 days. | observed. The inflammatory |
| 12.10A | Two of the animals had subcutaneous | reaction was also increased |
| | reddening and a muscle pale focus | (moderate to marked). |
| | whereas the third had a pale focus in the | For the rest of the animals, all |
| | muscle after 7 days. A pale focus in the | samples had the reactions |
| | muscle was noted for all animals after | lessened and a chronic |
| | 21 days. | mononuclear cell inflammatory |
| 12.10C | Subcutaneous reddening, gelatinous | response was observed. A |
| | texture and pale focus in the muscle | minimal to mild healing fibrosis |
| | were noted for one animal after 7 days. | and minimal to mild |
| | For the other two animals sacrificed | degeneration were observed. |
| | after 7 days a pale focus in the muscle | |
| | was seen. After 21 days one animal had | |
| | a pale focus in the muscle, the second | |
| | had a pale and raised focus in the | |
| | muscle whereas in the third a | |
| | subcutaneous edema was seen. | |
| Control | One animal had both subcutaneous | 7 days: |
| | reddening and pale focus in the muscle, | Minimal fibrosis was seen in 2 |
| | whereas the other two had one of the | out of 3 animals, as well as |
| | observations each after 7 days. After 21 | mild to moderate degeneration. |
| | days one animal had a pale focus in the | A mild to moderate mixed |
| | muscle, the second a subcutaneous | acute inflammatory reaction |
| | reddening and the third had NAD. | composed of mostly |
| | | mononuclear cells was |
| | | observed. |
| | | 21 days: |
| | | Moderate healing fibrosis and a |
| | | mild to moderate mostly |
| | | mononuclear cell inflammatory |
| | | reaction was observed. Minimal |
| | | to moderate degeneration was |
| | | observed and minimal necrosis |
| | | was noted in one of the 3 |
| | | animals. |

The implant materials were seen as multiple fragments around the surgical site due to tissue ingrowth and were not always readily observed, particularly 21 days after implantation. No material was observed after 21 days in 1 of 3 animals for alginate bolts 16.09B and in 2 of 3 animals for bolts 16.09D and the control. Little material was remaining in 1 of 3 animals for alginate bolts 12.10A and 12.10C.

The conclusions from the experiment are that under the conditions of the study, alginate bolts 16.09A, 16.09B, 16.09C, 16.09D and 12.10A were well tolerated, to a degree similar to the control material. Alginate bolt 12.10C caused a slightly greater, more prolonged reaction than the other alginate bolts, but was also considered to be well tolerated.

### Example 19

Pastes of alginate (PRONOVA UP MVG, DMC=88.7%) and a 1:1 mixture of chitosan (PROTASAN UP CL 110, DMC=91.4%) and hyaluronate (SODIUM HYALURONATE PHARMAGRADE 80, DMC=93.8%) were prepared as in Example 6. The chitosan and hyaluronate were premixed in dry condition before MilliQ water was added. The resulting paste had a calculated moisture content of 60%. The bolts were prepared in two different ways; 1) using the metal tube except that a 2-3 millimeter thick plug of non-swellable, non water-absorbable rubber was placed in each end of the metal tube to ensure that the paste was retained within the tube during compression. A metal plunger 5.8 millimeters in diameter was inserted into one end of the tube against the rubber plug and pushed in compression for 5 minutes using a vise; and 2) a metal tube was filled with paste and the paste was then pushed out of the tube using the metal plunger. The bolts were then air dried on the bench for at least two days.

The strength and size of the dried hyaluronate bolts were measured as described in Example 8. The alginate bolts were only measured in dry condition, while the 1:1 hyaluronate:chitosan bolts were measured both in dry condition and in rehydrated condition. The length and diameter of the bolts were measured using a caliper. The results are presented in Table XIII.

TABLE XIII
Results from size and breakage strength measurements of
bolts made by compression and bolts made without compression
(n = 3-5, average ± SD).

| | | Dry bolt | | | Rehydrated bolt | | | |
|---|---|---|---|---|---|---|---|---|
| | | | Maximum | | | Maximum | | |
| | | Force, | force, | Diameter, | Force, | force, | Diameter, | Swelling |
| Polymer | Compression | [g] | [g] | [mm] | [g] | [g] | [mm] | [%] |
| Hyaluronate:chitosan | Yes | 14200 ± 4300 | 19900 | 4.2 ± 0.2 | 2800 ± 800 | 3300 | 5.4 ± 0.3 | 32 ± 6 |
| (1:1) | | | | | | | | |
| Hyaluronate:chitosan | No | 17800 ± 3800 | 22600 | 4.1 ± 0.1 | 1200 ± 400 | 1500 | 5.6 ± 0.2 | 36 ± 3 |
| (1:1) | | | | | | | | |
| Alginate | Yes | 9200 ± 2100 | 11200 | 4.2 ± 0.1 | | | | |
| Alginate | No | 3000 ± 3400 | 8400 | 4.2 ± 0.2 | | | | |

Table XIII shows that compression of the paste gives stronger dry bolts where alginate is the only polymer. The compressed alginate bolts were more transparent and homogenous than the uncompressed alginate bolts both before and after drying. Another observation was that the uncompressed bolts had small cracks along the surface while the compressed bolts did not have such cracks. For the 1:1 mixture of hyaluronate and chitosan there is no significant effect on breakage strength on the dried bolts as a result of compression of the paste, but the compressed paste turns transparent which may indicate increased hydration of the polysaccharides in the blend. It seems that the anionic hyaluronate interacts with the cationic chitosan to form a more stable matrix of material and that breakage strength of a dry bolt is strong even without compression. Table XIII also shows that the breakage strength for compressed bolts of a 1:1 mixture of hyaluronate and chitosan is higher than for uncompressed bolts in a rehydrated condition, such as may occur upon implantation of the bolts into a body.

### Example 20

This example describes how to make a bolt from cross-linked calcium alginate fiber with a dry alginate gel coating. The example further shows the strength measurement of a dry bolt and a bolt that is partly hydrated in a model physiological solution.

A bolt was made from alginate fibers by winding a bundle of 5000 high-G alginate monofilaments up and down tightly around a needle (diameter: 1 mm, length: 5 cm). The windings were repeated about three times in each direction until the diameter of the bolt was about 5.6 mm. Then the bolt was placed in a 3% aqueous alginate solution (PRONOVA UP LVG, 1% viscosity: 44 mPas, F_{G}: ˜0.7) for 10 minutes. During this treatment it was seen that a gel layer was created around the bolt. This gel layer was created due to diffusion of calcium ions present in the fibers now available to gel the alginate solution surrounding the bolt. By this treatment the fibers on the surface of the bolt are partly dissolved and the bolt is coated with an alginate gel layer. To strengthen the coating layer the bolt was transferred into a gelling bath comprising 5% CaCl₂*2H₂O and 0.5% glycerol for 5 minutes. The needle was removed and the bolt was placed in the gelling bath. After gelling, the diameter of the bolt was about 7.4 mm. The bolt was dried under ambient conditions uncovered on the laboratory bench for at least two days. The diameter of the dry bolt was then about 6.2±0.9 mm. The dried bolt was about 24.9±3.6 mm long and weighed 0.89±0.05 grams (n=10).

To measure the dry strength of the bolt a Texture Analyzer (Stable Micro Systems (SMS), TA-XT2, load cell: 25 kg) and HDP/3PB Three Point Bend Rig was used with a base gap of 15 mm. The mode selected was: “Measure force in compression” and the pre-test speed and test speed were 0.5 mm/s and 0.2 mm/s, respectively. The distance was 10 mm and the trigger force was set to 5 g. The probe was adjusted to hit on the middle of the bolt between the two base legs upon which the bolt was placed. The force was applied vertically on the axis of the bolt. The measured breaking strength was 2480±360 g and the force applied per second before breakage was 240±80 g/s (n=5).

To see how the material swells upon hydration and how the strength suffers, the bolts were placed in 75 ml of Hanks' balanced salt solution (H8264, Sigma-Aldrich Chemie GmbH, Steinheim, Germany) Five bolts were placed in the same 100 ml weighing boat and kept in Hanks' at room temperature for two hours. The diameter and length of the bolts after two hours with swelling were 7.4±0.5 mm and 26.1±0.7 mm, respectively. The strength of the hydrated materials was tested with a Texture Analyzer (SMS, TA-XT21, load cell: 5 kg) and a HDP/BSG Blade Set with Guillotine. The mode selected was: “Measure force in compression” and pre-test speed and test speed were 0.5 mm/s and 0.25 mm/s, respectively. The distance was 10 mm and the trigger force was set to 1 g. The force was applied vertically on the axis of the bolt. The bolts had swelled 6±6% in the radial direction and 6±3% in the axial direction (n=5). The five bolts tested all survived the maximum load of the instrument of 6.4 kg which was obtained after the guillotine had traveled 4.1 mm±0.3 mm.

### Example 21

This example shows how to prepare a bolt from alginate fiber with a core of an extruded dried bolt made from a 1:1 blend of chitosan and hyaluronate. The example further demonstrates how swelling of the core material upon hydration in a model physiological solution is reduced by covering it with alginate fibers.

The extruded bolts were made by blending in a mortar dry powders of 3.21 g hyaluronate (SODIUM HYALURONATE PHARMA GRADE 80, Kibun Food Kemifa Co. Ltd., Tokyo, Japan, dry matter content (DMC): 93.5%) and 3.29 g chitosan (PROTASAN UP CL 210, NovaMatrix, FMC BioPolymer AS, Sandvika, Norway, DMC: 91.09%, degree of deacetylation: >95%). When the powders were blended 8.50 g MilliQ water was added and a homogeneous and hydrated rubber like paste was made with use of the mortar and hand kneading. The moisture content in the paste was 60%. Then the paste was pressed by hand into a metal tube with inner diameter of 6 mm and length of 40 mm. Rubber bolts (2-3 mm thick) were placed in each end of the metal tube and a metal plunger (diameter 5.8 mm) was placed at one end of the tube and the paste was then compressed for 5 minutes using a vice. The rubber bolts were placed at the ends of the tube to be able to exert more compressive force with the vice without extruding the paste. The bolts made from the paste were either dried uncovered under ambient conditions on the laboratory bench for at least two days or placed in a freezer at −18° C. overnight and then vacuum dried for one day. The freeze dried hyaluronate/chitosan bolts had an average diameter of 5.0±0.2 mm and an average density of 0.96±0.12 mg/cm³ (0.18±0.02 g/cm) (n=10). The air dried hyaluronate/chitosan bolts had an average diameter of 4.6±0.2 mm and an average density of 1.23±0.08 mg/cm³ (0.20±0.01 g/cm) (n=10).

The bolts were covered with 5000 high-G alginate monofilaments and by winding up and down tightly around a needle (diameter: 1 mm, length: 5 cm). The windings were repeated about two times in each direction around the bolts. The diameters of the extruded bolts covered by fiber were 6.4±0.3 mm and 6.9±0.3 mm for bolts with freeze dried and air dried cores, respectively. The weights of the extruded material and fiber were 0.71±0.10 g and 0.73±0.05 g for bolts with freeze dried and air dried cores, respectively.

Then the bolts were placed in a 3% aqueous alginate solution (PRONOVA UP LVG, 1% viscosity: 44 mPas, F_{G}: ˜0.7) for 10 minutes. During this treatment it was seen that a gel layer was created around the bolts. This gel layer was created due to diffusion of calcium ions present in the fibers now available to gel the alginate solution surrounding the bolts. By this treatment the fibers on the surface of the bolts are partly dissolved and the bolts were coated with an alginate gel layer. To strengthen the coating layer the bolt was transferred into a gelling bath comprising 5% CaCl₂*2H₂O and 0.5% glycerol for 5 minutes. The needle was removed and the bolt was placed in the gelling bath. The resulting thicknesses of the bolts were then 8.3±0.4 mm and 9.1±0.7 mm before drying for the bolts with freeze dried and air dried cores, respectively. After drying uncovered for two days under ambient conditions on the laboratory bench, the diameters and weights of the materials were 6.7±0.7 mm, 0.78±0.09 grams and 6.2±0.6 mm 0 81±0.09 grams for the bolts with freeze dried and air dried cores, respectively.

To measure the dry strength of the bolt a Texture Analyzer (Stable Micro Systems (SMS), TA-XT2, load cell: 25 kg) and HDP/3PB Three Point Bend Rig was used with a base gap of 15 mm. The mode selected was: “Measure force in compression” and the pre-test speed and test speed were 0.5 mm/s and 0.2 mm/s, respectively. The distance was 10 mm and the trigger force was set to 5 g. The probe was adjusted to hit on the middle of the bolt between the two base legs upon which the bolt was placed. The force was applied vertically on the axis of the bolt. The average breaking strength, maximum breaking strength and the force applied per second until breakage occurred, are summarized in Table XIV. The bolts without fibers were air dried and were not treated in an alginate solution and gelling bath.

TABLE XIV
Strength measurements of dry bolts (n = 4-5, ±SD).

| | Average | Maximum | Gradient, |
|---|---|---|---|
| | breaking | breaking | force/second |
| Bolt | strength, [g] | strength, [g] | [g/s] |
| Freeze dried hyaluronate: | 11 500 ± 5 700 | 19 400 | 1 140 ± 530 |
| chitosan (1:1) covered | | | |
| with alginate fibers | | | |
| Air dried hyaluronate: | 14 700 ± 4 800 | 20 700 | 850 ± 480 |
| chitosan (1:1) covered | | | |
| with alginate fibers | | | |
| Air dried hyaluronate: | 20 000 ± 9 000 | 36 800 | 2 970 ± 640 |
| chitosan (1:1) | | | |

The results presented in Table XIV do not show any significant differences between the materials, but indicate that a solid core material may provide a stiffer and stronger material. The force per second applied during measurement was higher for the material not covered with fibers. This is probably due to small amounts of air between the fibers and because compression of the fibers requires less force than was applied to the extruded bolt.

The materials were partly hydrated and the strength was measured as described above. All the bolts survived the maximum load of 6.4 kg. Table XV presents the swelling of the material and the distance the guillotine traveled before maximum load was applied.

TABLE XV
Strength measurements of hydrated materials (n = 4-5, ±SD).

| | Freeze dried | Air dried | Air dried |
|---|---|---|---|
| | hyaluronate:chitosan | hyaluronate:chitosan | hyaluronate: |
| | (1:1) covered with | (1:1) covered with | chitosan |
| Property | alginate fibers | alginate fibers | (1:1) |
| Radial | −0.5 ± 2.0 | 4 ± 5 | 36 ± 7 |
| swelling, | | | |
| [%] | | | |
| Axial | 6 ± 4 | 12 ± 13 | 7 ± 3 |
| swelling, | | | |
| [%] | | | |
| Average | >6 400 | >6 400 | 4 700 ± 1 200 |
| breaking | | | |
| strength, | | | |
| [g] | | | |
| Maximum | >6 400 | >6 400 | 6 000 |
| breaking | | | |
| strength, | | | |
| [g] | | | |
| Distance | 2.2 ± 0.3 | 2.5 ± 0.3 | 5.1 ± 0.7 |
| before | | | |
| maximum | | | |
| load, | | | |
| [mm] | | | |

The fibers reduced swelling in the radial direction, but since the fibers were not wound to cover the ends of the bolts, the bolts swelled more in the axial direction. For the bolts without fibers, the guillotine traveled longer before maximum load was applied. This indicates a more flexible material compared with the fiber coated materials. The use of fibers to cover a core made from an extruded biopolymer will reduce swelling and thereby also reduce hydration rate and degradation rate.

The foregoing examples have been presented for the purpose of illustration and description and are not to be construed as limiting the scope of the invention in any way. The scope of the invention is to be determined from the claims appended hereto.

1. An implantable degradable device suitable for use in tissue repair or reconstruction comprising at least one biopolymer, wherein said implantable device is pre-shaped by the application of pressure and said device comprises up to about 65% by weight of water, based on the total weight of the implantable degradable device.

2. The device of claim 1, wherein said biopolymer comprises a polysaccharide.

3. The device of claim 2, wherein said biopolymer comprises at least one of alginate, chitosan, and hyaluronate, modified polysaccharides, and mixtures thereof.

4. The device of claim 3, wherein said device is a screw, pin, bolt, anchor, rod, or plug.

5. The device of claim 1, wherein the biopolymer is not substantially ionically crosslinked.

6. The device of claim 1, wherein said device further comprises at least one material selected from the group consisting of plasticizers, at least one non-degradable biopolymer, uncrosslinked degradation controlling agents, imaging agents, pharmaceutically active agents, tissue regenerative agents, cell adhesion peptide sequences and growth factor agents.

7. The device of claim 2, wherein said device comprises at least one coating.

8. The device of claim 7, wherein said coating comprises at least one material selected from the group consisting of degradable biopolymers, imaging agents, pharmaceutically active agents, tissue regenerative agents, tissue adhesive agents, cell adhesion peptide sequences and growth factor agents.

9. The device of claim 8, wherein said coating comprises at least one material selected from the group consisting of sustained release agents, immediate release agents and delayed release agents.

10. The device of claim 1, wherein said device comprises a water:biopolymer ratio of 2:10 to 0.01:10.

11. The device of claim 1, wherein said device comprises a water:biopolymer ratio of 1.5:10 to 0.5:10.

12. The device of claim 1, wherein said biopolymer comprises at least one cationic biopolymer and at least one anionic biopolymer.

13. The device of claim 1, comprising at least one biopolymer fiber.

14. The device of claim 1, wherein at least a portion of said device is filled with a biopolymer hydrogel component.

15. The device of claim 14, wherein said biopolymer hydrogel component comprises a material selected from the group consisting of alginate, chitosan, hyaluronate, modified polysaccharides and mixtures thereof.

16. The device of claim 14, wherein said biopolymer hydrogel component further comprises at least one material selected from the group consisting of: imaging agents, pharmaceutically active agents, tissue regenerative agents, tissue adhesive agents, cell adhesion peptide sequences, growth factor agents and cells.

17. The device of claim 1, wherein said device has a density of from about 0.6 to about 1.5 mg/cm³.

18. Use of the device of claim 1 as a fixation device.

19. Use of the device of claim 1 as a drug delivery vehicle.

20. Use of the device of claim 1 as a scaffold for tissue growth.

21. A method of making an implantable degradable device suitable for use in tissue repair or reconstruction, as claimed in any one of claims 1-17 comprising the step of forming the device by application of pressure to a biopolymer-containing material.

22. The method of claim 21, wherein the pressure is applied to a biopolymer-containing material which contains at least 35% by weight of biopolymer, based on the total weight of the biopolymer-containing material.

23. The method of claim 22, wherein sufficient pressure is applied to a partially hydrated biopolymer-containing material to produce a homogeneous biopolymer-containing material that is more transparent after application of pressure than before application of pressure.

24. The method of claim 23, further comprising the step of drying said formed device by application of a drying step selected from air drying and freeze drying.

25. The method of claim 21, wherein said device is formed by at least one of molding, milling and extrusion.

26. The method of claim 21, further comprising the step of treating the formed device in an aqueous bath comprising at least one material selected from the group consisting of: plasticizers, degradable biopolymers, uncrosslinked degradation control agents, imaging agents, tissue adhesion agents, cell adhesion peptide sequences and growth factors, to form at least a partial coating on said device.

27. The method of claim 26, wherein said coating further comprises at least one material selected from the group consisting of sustained release agents, immediate relate agents and delayed release agents.

28. The method of claim 21, wherein said biopolymer-containing material comprises a cationic biopolymer and an anionic biopolymer and said method further comprises the step of determining a ratio of said cationic biopolymer to said anionic biopolymer contained in said biopolymer-containing material to control a rate of erosion, swelling and/or degradation of said device when implanted in a body.
